(19) United States
(12) Patent Application Publication
(10) Pub. No.: US 2024/0018509 A1
(43) Pub. Date: Jan. 18, 2024

(54) BINDING ASSAYS USING PHAGE DISPLAY TECHNOLOGY AND KITS THEREOF

(71) Applicant: Eurofins DiscoverX Corporation, San Diego, CA (US)

(72) Inventors: Jean Alexandre Bernatchez, San Diego, CA (US); Daniel Kelly Treiber, San Diego, CA (US); Ksenya Cohen Katsenelson, La Jolla, CA (US)

(73) Assignee: Eurofins DiscoverX Corporation, San Diego, CA (US)

(21) Appl. No.: 18/353,065

(22) Filed: Jul. 15, 2023

Related U.S. Application Data
(60) Provisional application No. 63/389,883, filed on Jul. 16, 2022.

(51) Int. Cl. C12N 15/10

(52) U.S. Cl. CPC C12N 15/1037

(57) ABSTRACT

The present invention provides methods and kits for identifying interactions between a test compound and a phage-displayed polypeptide, wherein the phage-displayed polypeptide is not a kinase but a protein domain of interest, such as an SH2 domain of interest. The protein domain of interest is displayed on the phage, and the interactions are evaluated in the presence of a reference ligand and in the presence and absence of the test compound.

### PRIORITY CLAIM AND CROSS-REFERENCE TO RELATED APPLICATIONS

This application claims the benefit and priority of the U.S. Provisional Application No. 63/389,883, filed on Jul. 16, 2022, the disclosure of which is incorporated by reference herein in its entirety, including all references and appendices cited therein, for all purposes.

### FIELD OF INVENTION

The subject matter provided herein relates to a competitive binding assay using phage display technology. Also provided herein are compositions, kits and methods of use thereof.

### BACKGROUND

The phage display system has been used traditionally to examine the interaction of the phage-displayed peptides with proteins or peptides. It has also been found to use phage display techniques to explore interactions between proteins or peptides and “small molecules”—i.e., typically synthetic organic molecules which may be useful as pharmaceutical compounds. This technique is described in PCT publication WO01/18234 published 15 Mar. 2001. There is a desire to use the technology to detect protein binders or protein binding partners for a candidate of choice for a drug target class. However, many proteins are still undruggable targets and thus cannot be used as protein binders or partners. Therefore, there is a need for a highly sensitive and selective method of protein binding for such undruggable targets.

Citation of documents herein is not intended as an admission that any is pertinent prior art. All statements as to the date or representation as to the contents of documents is based on the information available to the applicant and does not constitute any admission as to the correctness of the dates or contents of the documents.

### SPECIFIC PATENTS

U.S. Pat. No. 7,897,381, entitled “Uncoupling of DNA insert propagation and expression of protein for phage display” and owned by the present assignee provides a vector comprising a modified T7 phage genome.

U.S. Pat. No. 7,112,435, entitled “Uncoupling of DNA insert propagation and expression of protein for phage display” and owned by the present assignee provides an expression system comprising a modified T7 phage genome.

U.S. Pat. No. 7,833,741, entitled “Uncoupling of DNA insert propagation and expression of protein for phage display” and owned by the present assignee provides preparation of a heterologous polypeptide comprising a modified T7 phage genome.

U.S. Pat. No. 9,267,165, entitled “Assays and kits for detecting protein binding” and owned by the present assignee provides competitive binding assay using the phage technology.

### SUMMARY

The following is a summary of the invention to provide a basic understanding of some aspects of the invention. This summary is not intended to identify key/critical elements of the invention or delineate the invention's scope. Its sole purpose is to present some concepts of the invention in a simplified form as a prelude to the more detailed description that is presented later.

The present disclosure provides methods and kits for identifying interactions between a test compound and a phage-displayed polypeptide, such as a protein domain of interest and assessing the binding affinity of interactions of a test compound and a phage-displayed polypeptide in the presence of a reference ligand.

In various embodiments, the present disclosure provides a competitive binding assay using phage-display technology to assess the binding affinity of interactions, if any, of a test compound and a phage-displayed polypeptide in the presence of a reference ligand, wherein the reference ligand is immobilized on a solid support and the reference ligand is specific for the phage-displayed polypeptide.

In various other embodiments, the present disclosure provides a competitive binding assay to assess the binding affinity of interactions, if any, between one or more test compounds and a phage-displayed polypeptide in the presence of an immobilized reference ligand.

In many embodiments, the phage-displayed polypeptide may be exposed to an immobilized ligand, and the reaction is allowed to equilibrate. After washing to remove unbound phage, the reaction is contacted with a test compound which may elute bound phage particles from the immobilized ligand. The amount of eluted phage displaying the polypeptide as a function of the concentration of the test compound is used to determine the affinity(ies) of the phage-displayed polypeptide for the test compound. The immobilized ligand may be a reference ligand, wherein the ligand is specific for the phage-displayed polypeptide.

In various embodiments, the present disclosure also provides a method of identifying a test compound that binds to a phage-displayed SH2 domain of interest, comprising contacting a reference ligand immobilized on a solid support with a phage-displayed SH2 domain in the presence and absence of a test compound; incubating the immobilized reference ligand and the phage-displayed SH2 domain of interest under conditions allowing binding; removing unbound phage-displayed SH2 domain and detecting the phage-displayed SH2 domain bound to the immobilized reference ligand via qPCR amplification of a DNA amplicon located within the phage genome, whereby a decrease in the binding of the phage-displayed SH2 domain bound to the immobilized reference ligand in the presence of the test compound as compared to the absence of the test compound indicates that the test compound binds to the SH2 domain.

In various embodiments, the present disclosure further provides a method of identifying a test compound that binds to a phage-displayed human SH2 domain of interest, comprising: contacting a reference ligand immobilized on a solid support with a phage-displayed human SH2 domain in the presence and absence of a test compound; incubating the immobilized reference ligand and the phage-displayed human SH2 domain of interest under conditions allowing binding; removing unbound phage-displayed SH2 domain and detecting the phage-displayed human SH2 domain bound to the immobilized reference ligand via qPCR amplification of a DNA amplicon located within the phage genome, whereby a decrease in the binding of the phage-displayed human SH2 domain bound to the immobilized reference ligand in the presence of the test compound as compared to the absence of the test compound indicates that the test compound binds to the human SH2 domain.

In various embodiments, a reference ligand may be immobilized on a solid support such as a bead. The bead may be a magnetic bead or a streptavidin bead. In various other embodiments, the reference ligand is immobilized on streptavidin-coated magnetic beads, which may be further treated with biotinylated small molecule ligand to generate affinity resins for phage-displayed polypeptide assays, such as phage-displayed SH2 domain assays. The reference ligand may be a biotinylated ligand. In various other embodiments, a reference ligand may be a protein, wherein the protein is immobilized on a solid phase support bead wherein the protein will be cloned with an Avi tag at its N-terminus followed by purification and labelling with Desthio-Biotin, and then the labelled protein is immobilized to a magnetic streptavidin bead. In many embodiments, the reference ligand is specific or a known binding partner of the phage-displayed polypeptide, such as a reference ligand is specific for phage-displayed SH2 domain of interest. In many other embodiments, the reference ligand comprises a phosphopeptide sequence, such as a phosphorylated tyrosine sequence for SH2 binding.

In various embodiments, the ligand may be labelled with a tag for detection. The tag may be an enzymatic tag, a fluorescent tag, a spectroscopic tag, a reporter group, a fluorescent probe, or other detection probes.

In various embodiments, the phage-displayed polypeptide is a protein domain of interest, such as the SH2 domain, preferably a human SH2 domain. In various embodiments, the phage-displayed polypeptide is not a kinase polypeptide, and the phage-display polypeptide binds a specific peptide sequence such as a specific phosphopeptide sequence within the binding partner, wherein the binding partner is a compound, such as a test compound that binds to the phage-displayed polypeptide. Thus, the phage-displayed polypeptide, such as human SH2 domain binds to a specific phosphopeptide sequence within the test compound.

In many embodiments, the test compound is not previously known to bind to the phage-displayed protein domain, such as the SH2 domain, whereas the reference ligand is known to bind to the phage-displayed protein domain.

In various embodiments, the test compound may be a test molecule, a small molecule, a drug, a biologic, a protein, a polypeptide, a pharmaceutical drug, a candidate pharmaceutical, carbohydrate, or other compounds.

In many embodiments, the detection comprises quantifying the phage by qPCR of an amplicon located within the phage genome or via other detection techniques known for detecting fluorescent tag, spectroscopic tag, enzymatic probe and other detection methods.

In various embodiments, the disclosed assay may be conducted within a column, a single well plate, or a multi-well plate, wherein the solid support, such as a bead is in a column or a well of a multi-well plate.

The present disclosure also provides screening libraries of test compounds against the protein domain of interest, and once the binding interaction of interest has been identified, the test compound can be further evaluated individually.

In various embodiments, binding of a phage displayed polypeptide to the reference ligand occurs if the test compound does not bind to the phage-displayed polypeptide and wherein binding of the phage-displayed polypeptide to the reference ligand does not occur if the test compound does bind to the phage-displayed polypeptide. Further, the reference ligand is known to bind to the phage-displayed polypeptide, wherein said polypeptide is a protein domain of interest and the phage-displayed polypeptide binding to a phosphopeptide sequence within the reference ligand or the test compound.

The disclosure further provides methods of quantifying the interaction between a phage-displayed protein domain of interest and a test compound.

In various embodiments, provided here is a method of quantifying the interactions, comprising determining the concentration of a test compound at which about 50% of a phage-displayed polypeptide is bound to a reference ligand relative to the amount bound in the absence of said test compound, wherein the said concentration at which 50% of the phage-displayed polypeptide is bound to said reference ligand is identified as the value of the dissociation constant.

Also are included business methods for the pharmaceutical development of test compounds screened using the disclosed assay. Further, kits are provided for performing the assays as provided herein, comprising phage-displayed protein domain, such as human SH2 domain, and immobilized reference ligand, along with instructions for performing the methods described herein.

These and other features, aspects, and advantages of the present methods will become better understood with references to the following FIGURE and descriptions. This summary is an introduction of the concepts. Additional aspects and advantages of this invention will be apparent from the following detailed description of preferred embodiments, which proceeds with reference to the accompanying drawing.

### BRIEF DESCRIPTION OF THE FIGURE

The accompanying drawings, where like reference numerals refer to identical or functionally similar elements throughout the separate views, together with the detailed description below, are incorporated in and form part of the specification, and serve to illustrate further embodiments of concepts that include the claimed disclosure, and explain various advantages of those embodiments.

The methods disclosed herein have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

FIG. 1 shows a diagrammatic depiction of the disclosed binding assay to assess the binding affinity of interactions of a test compound and a phage-displayed protein domain of interest in the presence of a reference ligand.

### DETAILED DESCRIPTION

SH2 (Src Homology 2) domains are among the best characterized and most studied protein-protein interaction modules with an ability to bind and recognize sequences presenting a phosphorylated tyrosine and therefore are a key regulator of a plethora of physiological and molecular pathways possessing a fundamental role in cell signaling. SH2 domains, however, were long known to be undruggable targets and thus not a candidate of choice for a drug target class. The present invention provides an assay to evaluate binding partners for an SH2 domain, thus providing them as a candidate for further evaluation in drug discovery.

The disclosed binding assay employs a novel and proprietary competition binding assay to quantitatively measure interactions between a test compound and an SH2 domain of interest, such as a human SH2 domain of interest and evaluate the test compound as a binding partner for the SH2 domain of interest. This robust and reliable assay technology allows investigators to annotate a plurality of test compounds with accurate, precise and reproducible data.

The disclosed binding assay can screen a test compound to function as a binding compound or a binding partner for a protein domain of interest in the presence of a reference ligand. If the test compound is a binding partner of the protein domain of interest, the test compound competes with the reference ligand to bind to the protein domain of interest and if the test compound is not a binding partner of the protein domain of interest, there would be no competition between the reference ligand and the test compound to bind to the protein domain of interest.

The disclosed binding assay can screen test compounds to function as a binding partner for an SH2 domain of interest, such as a human SH2 domain, wherein the SH2 domain binds a specific peptide sequence within the binding partner, such as a specific phosphopeptide sequence within the test compound or the reference ligand. The disclosed method can also be used to screen test compounds via a high throughput assay wherein large libraries of compounds can be screened against a protein domain of interest and an immobilized ligand protein of choice.

The present disclosure further provides a binding assay comprising a phage-displayed polypeptide, wherein the polypeptide is a protein domain of interest, a test compound, and an immobilized ligand, wherein the protein domain of interest may be simultaneously exposed to one or more concentrations of the test compound and the immobilized ligand such that the binding of the phage-displayed protein domain of interest with the test compound decreases the binding of the phage-displayed protein domain with the immobilized ligand provided that the test compound is a binding partner for the phage-displayed protein domain of interest.

The present disclosure further provides a method of identifying a test compound that binds to a phage-displayed protein domain of interest, wherein the phage display protein domain of interest is not a kinase, comprising contacting a ligand immobilized on a solid support, with a phage-displayed protein domain of interest in the presence and absence of a test compound, incubating the immobilized ligand and the phage-displayed protein domain of interest under conditions allowing binding between the immobilized ligand and the phage-displayed protein domain of interest, wherein the phage-displayed protein domain of interest may bind a phosphopeptide sequence within the test compound; removing unbound phage-displayed protein domain of interest, and detecting the phage-displayed protein domain of interest bound to the immobilized ligand, whereby a decrease in the binding of the phage-displayed protein domain of interest to the immobilized ligand in the presence of the test compound as compared to the absence of the test compound indicates that the test compound binds to the protein domain of interest. In a preferred embodiment, the protein domain of interest may be an SH2 domain of interest, such as a human SH2 domain, wherein the SH2 domain binds to a phosphopeptide sequence within its binding partner, which may be a reference ligand or a test compound. The disclosed method may also be used to detect binding in the presence and absence of more than one test compound.

The present disclosure also provides a method of identifying a test compound binding to a phage-displayed human SH2 domain of interest, comprising contacting a ligand immobilized on a solid support, with a phage-displayed human SH2 domain in the presence and absence of a test compound, incubating the immobilized ligand and the phage-displayed human SH2 domain of interest under conditions allowing binding wherein the phage-displayed human SH2 domain of interest is simultaneously exposed to one or more concentrations of the test compound and the immobilized ligand and wherein the test compound is not previously known to bind to the phage-displayed human SH2 domain of interest, removing unbound phage-displayed human SH2 domain, and detecting the phage-displayed human SH2 domain bound to the immobilized ligand via qPCR amplification of a DNA amplicon located within the phage genome, whereby a decrease in the binding of the phage-displayed human SH2 domain bound to the immobilized ligand in the presence of the test compound as compared to the absence of the test compound indicates that the test compound binds to the human SH2 domain of interest. The method further comprises contacting the ligand immobilized on a solid support in the presence and absence of more than one test compound. The phage displayed human SH2 domain binds a phosphopeptide sequence, such as a phosphorylated tyrosine sequence within the test compound or the reference ligand.

In many embodiments, the test compound may be a competitor against the reference ligand for binding to the phage-displayed protein domain.

In the disclosed method, the phage-displayed polypeptide is exposed to an immobilized ligand and the reaction is allowed to equilibrate. After washing to remove unbound phage, the reaction is contacted with a test compound which may elute bound phage particles from the immobilized ligand. The amount of eluted phage displaying the polypeptide as a function of the concentration of the test compound is used to determine the affinity(ies) of the protein domain for the test compound. The amount of phage eluted is quantified via qPCR amplification of a DNA amplicon located within the phage genome.

The disclosed assay may therefore detect binding affinities of one or more than one test compounds with the phage-displayed protein domain of interest, such as a phage-displayed SH2 domain of interest. The binding between the phage-displayed SH2 domain of interest and the test compound may be reflected as a binding constant, which may be expressed as a dissociation constant or an association constant. The binding constants may be used to identify the test compound (s) as specific and/or selective for the SH2 domain tested or relatively non-specific and/or non-selective due to non-specific interactions with the SH2 domain.

The invention is also directed to a method to apply phage display technology to simultaneously contact a phage-displayed polypeptide with a reference ligand immobilized on a solid support and a test compound at a sufficient concentration to decrease the binding of the displayed polypeptide to the reference ligand. The concentration of the test compound necessary to diminish binding of the displayed polypeptide from the reference ligand may be used to determine a dissociation constant (Kd) for the test compound. The resulting Kd values may be compared to identify the test compound as specific and/or selective for the SH2 domain, particularly the human SH2 domain.

Therefore, the test compound that binds the protein domain of interest, such as SH2 domain of interest may be classified as strong, moderate and weak affinity binders of the protein domain of interest. Strong affinity binders may be determined by using a high concentration of the test compound wherein the test compound successfully compete with the immobilized reference ligand for binding to the phage-displayed protein domain of interest. Moderate and low affinity binders can be determined by using a medium concentration of a test compound to release a moderate affinity protein domain of interest from the solid support while not being sufficiently high concentration to release a low affinity polypeptide from the solid support.

The present disclosure further provides a method to determine the dissociation constant of a test compound with a protein domain of interest, comprising the assessment of the binding of the phage-displayed polypeptide to a ligand in the presence of various concentrations of a test compound. The number of phages bound to the immobilized ligand as a function of the test compound concentration may be plotted on a graph, and the Kd is calculated by fitting the curve to an appropriate binding equation. In some embodiments, the concentration of the test compound at which binding of the phage-displayed member to the ligand is reduced by 50% is equal to the Kd for the interaction between the displayed protein and the test compound.

The phage bound to the solid support at one or more concentrations of the test compound can be eluted, preferably after removal of unbound phage, and enumerated by standard phage tittering methods. A decrease in the amount of phage bound to the solid support in the presence of the test compound identifies the test compound as a binder of the SH2 domain, such as a binder of the human SH2 domain.

Further, phage displaying a polypeptide may be bound by low or high concentrations of a test compound that has a high degree of affinity for the phage-displayed polypeptide. High concentrations of a test compound with low or moderate affinity for the polypeptide are needed to prevent phage association with the reference ligand. Therefore, phage-displayed polypeptide that binds to the reference ligand despite exposing or incubating the phage-displayed polypeptide with a high concentration of the test compound may be identified as displaying a polypeptide that has no or minimal interactions with the test compound. Phage-displayed polypeptides which are detectably bound to the immobilized reference ligand in the absence of a test compound, but which are no longer detectable even at low concentrations of the compound are identified as high affinity binders to the said test compound.

Additionally, the test compounds may be used as described above to identify other proteins or peptides as potential targets of a test compound. A disease condition or indication that is associated with an identified protein or peptide may thus be one that can also be treated clinically by use of said test compound. Notably, the dissociation constant that can be determined for said test compound relative to said identified protein or peptide may also be used to provide an estimation of the in vivo concentration of said test compound for use during therapy.

The invention also provides for the formulation of dissociation constant information into a database or other tabular form for ease of use and subsequent analysis.

As used herein, a “test compound” refers to the chemical entities such as, but not limited to, a test molecule, protein, organic or inorganic molecule, carbohydrate, or other compound to which a polypeptide is tested for binding. A “test molecule” of the invention includes pharmaceuticals and candidate pharmaceuticals which are natural products or which are prepared synthetically. Non-limiting examples include polyketides, steroids, the compounds found in the U.S. Pharmacopoeia, and the products of combinatorial chemical synthesis. Candidate pharmaceuticals include molecules for which no function is known, but which have structural similarity to known compounds with one or more known functions. “Polypeptide” refers to any protein or peptide, naturally occurring or synthetic (including fragments, portions, and mutants of a protein or peptide) composed of amino acids linked by peptide (amide) bonds. The amino acids may be naturally occurring or synthetic, including D- and L-forms of amino acids.

The polypeptide, preferably displayed on a phage particle, is exposed to a “reference ligand” that is preferably immobilized on a solid support. Immobilization of the reference ligand may be by a variety of means, and standard means of covalently or non-covalently coupling a molecule to solid supports are well known in the art. Non-limiting examples include the use of linker molecules, crosslinkers such as glutaraldehyde, and biotin/avidin interactions. An example of the latter is with the use of biotin covalently coupled to a compound and avidin bound to a solid support. The solid support itself can take any convenient form, typically a culture dish or plate or bottle, a well of a multi-well culture dish or plate, a bead, a column containing particles to which a molecule is immobilized, or a planar surface containing the immobilized compound. Other non-limiting examples of a solid support include agarose, polystyrene or other polyvinyl compounds, and magnetic beads.

The phage-displayed protein or peptide is produced as a fusion polypeptide with a coat protein characterizing the phage. The displayed, non-phage protein can be coupled to the C-terminus or the N-terminus of the coat protein characteristic of the phage. A variety of phages may be used in the practice of the invention, including lytic bacteriophage vectors (e.g., lambda, T4 and T7), filamentous phage (e.g., M13), and other vector means including viruses.

In one aspect, the present invention utilizes phage particles displaying known individual protein members of a group or family. Homogenous phage particles displaying a protein are exposed to, or contacted with, a reference ligand immobilized on a solid support, such as a magnetic bead. Thus, each assay well contains an immobilized reference ligand that binds to phage particles that display a protein member of a group or family. The phage particles in each well are also exposed to, or contacted with, a concentration of a test compound. The phage is prevented from binding the solid support when there is binding to the test compound. The number of phages remaining bound to the solid support at various concentrations of the test compound is determined by eluting the support-bound phage and then performing standard phage titering assays (plaque assays) or quantitative PCR-based methods, such as qPCR.

In some embodiments, in a microtiter plate or dish format, all wells may contain the same immobilized reference ligand while each column of wells contains phage particles displaying the same protein domain of interest. Each row of wells contains a different test compound and/or a different concentration of a test compound.

The ligand may optionally be immobilized, such as by attachment to a solid support, e.g., a bead surface. The ligand may also be optionally labeled with, for example, fluorescence and/or spectroscopic tags. The amount of phage particles bound to or displaced from the ligand relative to the concentration of the test compound permits the determination of the affinity of the interaction between the test compound and the protein domain of interest. Alternatively, the reference ligand may be labeled with a reporter group, such as a fluorescent probe, that permits alternative readouts of the interaction between the protein domain of interest and the ligand. Fluorescence polarization is a non-limiting example of a method that could be used to detect interactions between the labeled ligand and the protein domain at various concentrations of the test compound. The ligand may be a small molecule, a drug, a biologic, a protein, carbohydrate, a pharmaceutical drug, a candidate pharmaceutical or other compounds.

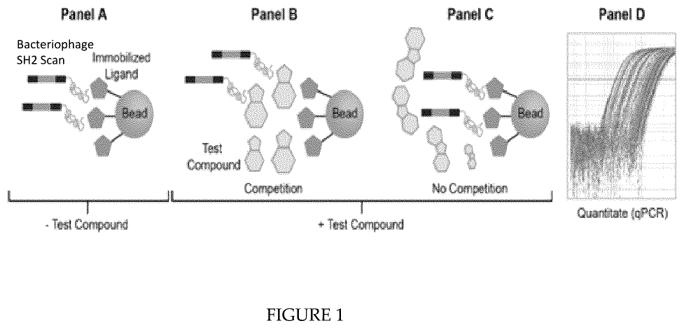

FIG. 1 shows the principle of the disclosed competitive binding assay wherein the ligand of choice is immobilized (Panel A) on a solid surface such as a streptavidin-coated magnetic bead surface and exposed to a phage-displayed SH2 domain of interest. The immobilized ligand and the phage displayed SH2 domain is further incubated with a test compound (Panel B), wherein if the test compound binds with the SH2 domain to form a complex, the SH2 domain does not bind to the immobilized ligand such that a lower signal will be generated (Panel C). If the test compound does not bind with the SH2 domain, a higher signal will be detected in the qPCR stage (Panel D), showing that the test compound is not a binding partner for the SH2 domain of interest.

Therefore, test compounds that bind the SH2 domain of interest and prevent the SH2 domain from binding to the immobilized ligand will reduce the amount of SH2 domain captured on the solid support (Panel B) compared to the baseline signal as shown in Panel D. Conversely, test compounds that do not bind the SH2 domain of interest have no effect on the amount of SH2 domain captured on the solid support (Panel C). Screening “hits” are identified by measuring the amount of SH2 domain captured in test versus control samples by using a quantitative, precise and ultra-sensitive qPCR method that detects PCR amplicons located within the phage genome (Panel D).

### EXPERIMENTATION

SH2 domain-tagged T7 phage strains were prepared in an E. coli host derived from the BL21 strain. E. coli were grown to log-phase, infected with T7 phage, and incubated with shaking at 32° C. until lysis. The lysates were centrifuged and filtered to remove cell debris. Streptavidin-coated magnetic beads were treated with biotinylated small molecule ligands for 30 minutes at room temperature to generate affinity resins for SH2 domain assays. The liganded beads were blocked with excess biotin and washed with blocking buffer (SeaBlock (Pierce), 1% BSA, Tween 20.1 mM DTT) to remove unbound ligand and to reduce non-specific binding. Binding reactions were assembled by combining SH2 domains, liganded affinity beads, and test compounds in 1× binding buffer (10 mM HEPES, 50 mM NaCl, 1 mM EDTA, 0.01% Tween 20, 6 mM DTT). Test compounds were prepared as 100× stocks in 100% DMSO. Kds were determined using an 11-point 3-fold compound dilution series with three DMSO control points. All compounds for Kd measurements are distributed by acoustic transfer (non-contact dispensing) in 100% DMSO. The compounds were then diluted directly into the assays such that the final concentration of DMSO was 1%. All reactions were performed in a polypropylene 384-well plate. Each was a final volume of 0.02 ml. The assay plates were incubated at room temperature with shaking for 1 hour, and the affinity beads were washed with wash buffer (10 mM HEPES, 50 mM NaCl, 1 mM EDTA, Tween 20). The beads were then re-suspended in elution buffer (10 mM HEPES, 50 mM NaCl, 1 mM EDTA, 0.01% Tween 20, 20 mM sodium phenyl phosphate) and incubated at room temperature with shaking for 30 minutes. The SH2 domain concentration in the eluates was measured by qPCR.

The invention is preferably practiced by using phage genomes that are conservatively modified to contain heterologous polypeptide encoding sequences fused to a phage surface protein. The modified phage genomes preferably retain the regulatory and coding sequences found therein. Preferred phage genomes for the practice of the invention are those of lytic phages, not limited to T7, T4, T3 and lambda phage, and filamentous phages.

Aspects of the methods include using a vector comprising a modified T7 phage genome, which is extensively described in the aforementioned reference U.S. Pat. No. 7,897,381 issued on Mar. 1, 2011, whose method section is specifically incorporated by reference as if set forth in its entirety herein.

Aspects of the method include using an expression system comprising a modified T7 phage genome, which is extensively described in the aforementioned reference U.S. Pat. No. 7,112,435 issued on Sep. 26, 2006, whose method section is specifically incorporated by reference as if set forth in its entirety herein.

Aspects of the method include the preparation of a heterologous polypeptide comprising a modified T7 phage genome, which is extensively described in the aforementioned reference U.S. Pat. No. 7,833,741 issued on Sep. 26, 2006, whose method section is specifically incorporated by reference as if set forth in its entirety herein.

Aspects of the method include a competitive binding assay using phage display technology comprising a test compound, a phage-displayed polypeptide, and a reference ligand which is extensively described in the aforementioned reference U.S. Pat. No. 9,267,165 issued on Feb. 23, 2016, whose method section is incorporated by reference as if set forth in its entirety herein.

The invention may also be practiced via sequential or parallel contacting of displayed polypeptides immobilized on a solid support via binding to a reference ligand (in a solution containing no test compound) with 1) a solution containing a low concentration of test compound, 2) a solution containing a medium concentration of test compound, and 3) a solution containing a high concentration of test compound permitting the sequential release of polypeptides with high, moderate, and low affinities, respectively, for said test compound.

Therefore, the methods as disclosed in this invention may be used to determinate relative strength of binding of a test compound to a protein domain of interest and used as a screening tool to identify one or more test compounds as binders to the protein domain of interest.

The disclosure also provides kits for assessing a potential interaction between a phage-displayed polypeptide and a test compound, comprising said phage-displayed polypeptide and a reference ligand, wherein the reference ligand is capable of binding said phage-displayed polypeptide. In various embodiments, the phage-displayed polypeptide is a phage-displayed protein domain of interest, such as phage-displayed SH2 domain of interest and the SH2 domain binds to a phosphopeptide sequence within the reference ligand or the test compound.

In the description, for purposes of explanation and not limitation, specific details are set forth, such as particular embodiments, systems, methods, etc. in order to provide a thorough understanding of the present technology. However, it will be apparent to one skilled in the art that the present technology may be practiced in other embodiments that depart from these specific details.

While the presently disclosed systems and methods are susceptible of embodiment in many different forms, there are shown in the drawings and will herein be described in detail several specific embodiments with the understanding that the present disclosure is to be considered as an exemplification of the principles of the presently disclosed systems and methods and is not intended to limit the disclosure systems and methods to the embodiments illustrated.

While specific embodiments of, and examples for, the system is described above for illustrative purposes, various equivalent modifications are possible within the scope of the system, as those skilled in the relevant art will recognize. For example, while processes or steps are presented in a given order, alternative embodiments may perform routines having steps in a different order, and some processes or steps may be deleted, moved, added, subdivided, combined, and/or modified to provide alternative or sub-combinations. Each of these processes or steps may be implemented in a variety of different ways. Also, while processes or steps are at times shown as being performed in series, these processes or steps may instead be performed in parallel or may be performed at different times.

While various embodiments have been described above, it should be understood that they have been presented by way of example only and not limitation. The descriptions are not intended to limit the scope of the present technology to the particular forms set forth herein. To the contrary, the present descriptions are intended to cover such alternatives, modifications, and equivalents as may be included within the spirit and scope of the present technology as appreciated by one of ordinary skill in the art. Thus, the breadth and scope of a preferred embodiment should not be limited by any of the above-described exemplary embodiments.

What is claimed is:

1. A method of screening a test compound that binds to a phage-displayed protein domain of interest, comprising:
contacting a reference ligand immobilized on a solid support, with a phage-displayed protein domain of interest in the presence and absence of the test compound, wherein the phage-displayed protein domain of interest is not a kinase;
incubating the immobilized reference ligand and the phage-displayed protein domain of interest under conditions allowing binding between the immobilized reference ligand and the phage-displayed protein domain of interest in the presence and absence of the test compound, wherein the phage-displayed protein domain of interest binds a specific peptide sequence within the test compound or the immobilized reference ligand;
removing the unbound phage-displayed protein domain of interest and detecting the phage-displayed protein domain of interest bound to the immobilized reference ligand, whereby a decrease in the binding of the phage-displayed protein domain of interest to the immobilized reference ligand in the presence of the test compound as compared to the absence of the test compound indicates that the test compound binds to the protein domain of interest.

2. The method of claim 1, wherein the protein domain of interest is an SH2 domain of interest.

3. The method of claim 2, wherein the SH2 domain of interest is a human SH2 domain.

4. The method of claim 1, wherein the test compound is a small molecule.

5. The method of claim 1, wherein the test compound is a candidate pharmaceutical.

6. The method of claim 1, wherein the phage-displayed protein domain of interest binds a specific phosphopeptide sequence within the test compound or the immobilized reference ligand.

7. The method of claim 1, wherein the detection comprises quantifying the phage by quantitative polymerase chain reaction.

8. The method of claim 1, wherein the reference ligand is a protein, wherein the protein is immobilized on a solid phase support bead.

9. The method of claim 1, wherein the method further comprises contacting the reference ligand immobilized on a solid support in the presence and absence of more than one test compound.

10. The method of claim 1, wherein the protein domain of interest is simultaneously exposed to one or more concentrations of the test compound.

11. The method of claim 1, wherein the test compound is not previously known to bind to the phage-displayed protein domain of interest.

12. A method of identifying a test compound that binds to a phage-displayed SH2 domain of interest, comprising:
contacting a reference ligand immobilized on a solid support, with a phage-displayed SH2 domain of interest in the presence and absence of the test compound;
incubating the immobilized reference ligand and the phage-displayed SH2 domain of interest under conditions allowing binding;
removing unbound phage SH2 domain of interest and detecting the phage-displayed SH2 domain of interest bound to the immobilized reference ligand via qPCR amplification of a DNA amplicon located within the phage genome, whereby a decrease in the binding of the phage-displayed SH2 domain of interest bound to the immobilized reference ligand in the presence of the test compound as compared to the absence of the test compound indicates that the test compound binds to the SH2 domain of interest.

13. The method of claim 12, wherein the SH2 domain is a human SH2 domain.

14. The method of claim 12, wherein the test compound is a small molecule.

15. The method of claim 12, wherein the SH2 domain binds a specific peptide sequence within the test compound or the reference ligand.

16. The method of claim 15, wherein the specific peptide sequence within the test compound or the reference ligand is a specific phosphopeptide sequence.

17. The method of claim 12, wherein the reference ligand is a protein, wherein the protein is immobilized on a solid phase support bead.

18. The method of claim 12, wherein the method further comprises contacting the reference ligand immobilized on a solid support in the presence and absence of more than one test compound.

19. The method of claim 12, wherein the protein domain is simultaneously exposed to one or more concentrations of the test.

20. The method of claim 12, wherein the immobilized ligand is known to bind to the phage-displayed SH2 domain of interest.
